SEQUENCE LISTING (13 sequences)

SEQ ID NO: 1 - protein, 362 aa, Human polyomavirus 1
MAPTKRKGECPGAAPKKPKEPVQVPKLLIKGGVEVLEVKTGVDAITEVECFLNPEMGDPDENLRGFSLKLSAENDFSSDSPERKMLPCYSTARIPLPNLNEDLTCGNLLMWEAVTVQTEVIGITSMLNLHAGSQKVHEHGGGKPIQGSNFHFFAVGGDPLEMQGVLMNYRTKYPEGTITPKNPTAQSQVMNTDHKAYLDKNNAYPVECWIPDPSRNENTRYFGTFTGGENVPPVLHVTNTATTVLLDEQGVGPLCKADSLYVSAADICGLFTNSSGTQQWRGLARYFKIRLRKRSVKNPYPISFLLSDLINRRTQRVDGQPMYGMESQVEEVRVFDGTERLPGDPDMIRYIDKQGQLQTKML

SEQ ID NO: 2 - protein, 362 aa, Human polyomavirus 1
MAPTKRKGECPGAAPKKPKEPVQVPKLLIKGGVEVLEVKTGVDAITEVECFLNPEMGDPDDNLRGYSLKLTAENAFDSDSPDKKMLPCYSTARIPLPNLNEDLTCGNLLMWEAVTVKTEVIGITSMLNLHAGSQKVHENGGGKPVQGSNFHFFAVGGDPLEMQGVLMNYRTKYPQGTITPKNPTAQSQVMNTDHKAYLDKNNAYPVECWIPDPSRNENTRYFGTYTGGENVPPVLHVTNTATTVLLDEQGVGPLCKADSLYVSAADICGLFTNSSGTQQWRGLARYFKIRLRKRSVKNPYPISFLLSDLINRRTQKVDGQPMYGMESQVEEVRVFDGTEQLPGDPDMIRYIDRQGQLQTKMV

SEQ ID NO: 3 - protein, 362 aa, Human polyomavirus 1
MAPTKRKGECPGAAPKKPKEPVQVPKLLIKGGVEVLEVKTGVDAITEVECFLNPEMGDPDDHLRGYSQHLSAENAFDSDSPDKKMLPCYSTARIPLPNLNEDLTCGNLLMWEAVTVKTEVIGITSMLNLHAGSQKVHENGGGKPVQGSNFHFFAVGGDPLEMQGVLMNYRTKYPQGTITPKNPTAQSQVMNTDHKAYLDKNNAYPVECWIPDPSKNENTRYFGTYTGGENVPPVLHVTNTATTVLLDEQGVGPLCKADSLYVSAADICGLFTNSSGTQQWRGLARYFKIRLRKRSVKNPYPISFLLSDLINRRTQKVDGQPMYGMESQVEEVRVFDGTEQLPGDPDMIRYIDRQGQLQTKMV

SEQ ID NO: 4 - protein, 362 aa, Human polyomavirus 1
MAPTKRKGECPGAAPKKPKEPVQVPKLLIKGGVEVLEVKTGVDAITEVECFLNPEMGDPDNDLRGYSLRLTAETAFDSDSPDRKMLPCYSTARIPLPNLNEDLTCGNLLMWEAVTVKTEVIGITSMLNLHAGSQKVHENGGGKPIQGSNFHFFAVGGDPLEMQGVLMNYRTKYPEGTVTPKNPTAQSQVMNTDHKAYLDKNNAYPVECWIPDPSRNENTRYFGTYTGGENVPPVLHVTNTATTVLLDEQGVGPLCKADSLYVSAADICGLFTNSSGTQQWRGLPRYFKIRLRKRSVKNPYPISFLLSDLINRRTQRVDGQPMYGMESQVEEVRVFDGTEQLPGDPDMIRYIDRQGQLQTKMV

SEQ ID NO: 5 - protein, 354 aa, Human polyomavirus 2
MAPTKRKGERKDPVQVPKLLIRGGVEVLEVKTGVDSITEVECFLTPEMGDPDEHLRGFSKSISISDTFESDSPNKDMLPCYSVARIPLPNLNEDLTCGNILMWEAVTLKTEVIGVTTLMNVHSNGQATHDNGAGKPVQGTSFHFFSVGGEALELQGVVFNYRTKYPDGTIFPKNATVQSQVMNTEHKAYLDKNKAYPVECWVPDPTRNENTRYFGTLTGGENVPPVLHITNTATTVLLDEFGVGPLCKGDNLYLSAVDVCGMFTNRSGSQQWRGLSRYFKVQLRKRRVKNPYPISFLLTDLINRRTPRVDGQPMYGMDAQVEEVRVFEGTEELPGDPDMMRYVDRYGQLQTKML

SEQ ID NO: 6 - protein, 10 aa, synthetic construct
  REGION 1..10: note = Made in Lab - heavy CDR1 from MAU868 monoclonal antibody
GFTFNNYWMT

SEQ ID NO: 7 - protein, 17 aa, synthetic construct
  REGION 1..17: note = Made in Lab - heavy CDR2 from MAU868 monoclonal antibody
NIKKDGSEKYYVDSVRG

SEQ ID NO: 8 - protein, 11 aa, synthetic construct
  REGION 1..11: note = Made in Lab - heavy CDR3 from MAU868 monoclonal antibody
VRSGRYFALDD

SEQ ID NO: 9 - protein, 11 aa, synthetic construct
  REGION 1..11: note = Made in Lab - light CDR1 from MAU868 monoclonal antibody
GGDNIGSRPVH

SEQ ID NO: 10 - protein, 7 aa, synthetic construct
  REGION 1..7: note = Made in Lab - light CDR2 from MAU868 monoclonal antibody
DDSNRPS

SEQ ID NO: 11 - protein, 10 aa, synthetic construct
  REGION 1..10: note = Made in Lab - light CDR3 from MAU868 monoclonal antibody
QVWSSSTDHP

SEQ ID NO: 12 - protein, 450 aa, synthetic construct
  REGION 1..450: note = Made in Lab - heavy chain variable region from MAU868 monoclonal antibody
QVQLVESGGTLVQPGGSLRLSCAASGFTFNNYWMTWVRQAPGKGLEWVANIKKDGSEKYYVDSVRGRFTISRDNAKNSLFLQMNSLRPEDTAVYFCATVRSGRYFALDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 213 aa, synthetic construct
  REGION 1..213: note = Made in Lab - light chain variable region from MAU868 monoclonal antibody
QSVLTQPPSVSVAPGKTARITCGGDNIGSRPVHWYQQKPGQAPILVVYDDSNRPSGIPERFSGSNSGNTATLTISRVEAGDEADYYCQVWSSSTDHPFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2026/0265346 A1
(43) Pub. Date: Sep. 10, 2026

(54) DOSING OF POLYOMAVIRUS NEUTRALIZING ANTIBODIES

(71) Applicants: Vera Therapeutics, Inc., Brisbane, CA (US); Novartis AG, Basel (CH)

(72) Inventors: Johanna R. ABEND, Emeryville, CA (US); Mark KNAPP, Emeryville, CA (US); Steven J. KOVACS, East Hanover, NJ (US); Amy K. PATICK, Alameda, CA (US); Elisabetta TRAGGIAI, Basel (CH)

(21) Appl. No.: 19/454,223

(22) Filed: Jan. 20, 2026

Related U.S. Application Data
(63) Continuation of application No. 18/064,081, filed on Dec. 9, 2022, now abandoned.
Application No. 18/064,081 is a continuation of application No. PCT/US2021/036923, filed on Jun. 11, 2021.
(60) Provisional application No. 63/038,433, filed on Jun. 12, 2020.

(51) Int. Cl. C07K 16/084; A61K 39/00

(52) U.S. Cl. CPC C07K 16/084; A61K 2039/505; A61K 2039/545; C07K 2317/565; C07K 2317/70

(57) ABSTRACT

Provided are dosing regimens of polyomavirus neutralizing antibodies and related methods and pharmaceutical compositions for treating polyomavirus infections.

### CROSS-REFERENCE TO RELATED APPLICATIONS

This application is a continuation of U.S. application Ser. No. 18/064,081, filed Dec. 9, 2022, which is a continuation of International Application No. PCT/US2021/036923, filed Jun. 11, 2021, which claims the benefit of U.S. Provisional Application No. 63/038,433, filed on Jun. 12, 2020, each of which is incorporated by reference herein in its entirety for all purposes.

### REFERENCE TO AN ELECTRONIC SEQUENCE LISTING

The content of the electronic sequence listing (VETH_014_02US_SeqList_ST26.xml; Size: 18,094 bytes; and Date of Creation: Jan. 20, 2026) is herein incorporated by reference in its entirety.

### BACKGROUND

### Technical Field

The present disclosure relates to dosing regimens of polyomavirus neutralizing antibodies and related methods and pharmaceutical compositions for treating polyomavirus infections.

### Description of the Related Art

Polyomaviruses such as BK virus and JC virus are ubiquitous with a worldwide prevalence of about 75-90%. Following initial infection, the viruses remain latent in most people, but reactivation may occur in immunocompromised patients, including transplant patients who are immunocompromised from immunosuppressive therapy used to prevent transplant rejection.

For example, in kidney transplant recipients, reactivation of BK virus can lead to BK viremia, which can result in BKV-associated nephropathy—a leading cause of irreversible kidney damage and graft failure. The current standard of care is to reduce immunosuppression, which carries significant risk of active immune system rejection of the transplanted organ. Also, reactivation of BK virus in hematopoietic cell transplant (HCT) recipients may cause BKV-associated hemorrhagic cystitis, an intensely painful condition that can last weeks to months and cause prolonged hospitalizations. Current care for hemorrhagic cystitis is generally supportive, including narcotic analgesics, hydration, and diuresis. Many patients also require bladder irrigation, clot evacuation, blood transfusion, stenting, and nephrostomy. There are currently no approved treatments for BK virus infections.

MAU868 is a human monoclonal antibody (immunoglobulin G, IgG1/λ isotype subclass) that potently neutralizes all four BKV serotypes (see, for example, WO 2017/046676, as P8D11). It recognizes a conformational epitope on the viral capsid protein (VP1), which is responsible for binding to and facilitating infection of new target cells. MAU868 also has neutralizing activity against the closely-related JC virus, the cause of progressive multifocal leukoencephalopathy. This neutralizing antibody could minimize complications in renal transplant patients by avoiding the most common cause of viral allograft loss, and also significantly simplify the immunosuppressive therapy regimen that clinicians currently face. Similarly, treatment or prevention of BK virus-related hemorrhagic cystitis in HCT recipients could eliminate or reduce a complication associated with significant morbidity and mortality and acute graft-versus-host disease.

However, there is a need in the art to optimize the dosing of polyomaviruses neutralizing antibodies.

### BRIEF SUMMARY

Embodiments of the present disclosure include a dosing regimen for treatment of a BK or JC polyomavirus infection in a human subject in need thereof, comprising
(a) parenterally administering to the subject a dosage of an antibody, or an antigen-binding fragment thereof, which specifically binds to a VP1 protein of the polyomavirus;
(b) measuring serum or tissue concentration of the antibody, or antigen-binding fragment thereof, in the subject; and
(c) administering a further dosage of the antibody, or antigen-binding fragment thereof, before the serum or tissue trough concentration (C_{trough}) falls below about 3-860 μg/mL;
wherein the dosing regimen maintains the serum or tissue concentration of the antibody, or antigen-binding fragment thereof, above the C_{trough} throughout the treatment.

In some embodiments:
(i) the C_{trough} in (c) is for plasma, and the dosing regimen comprises administering the further dosage before the plasma C_{trough} falls below about 150-860 μg/mL;
(ii) the C_{trough} in (c) is for renal tissue, and the dosing regimen comprises administering the further dosage before the C_{trough} in renal tissue falls below about 23.5-120 μg/mL; or
(iii) the C_{trough} in (c) is for bladder tissue, and the dosing regimen comprises administering the further dosage before the C_{trough} in bladder tissue falls below about 3-10 μg/mL.

In some embodiments, the antibody, or antigen-binding fragment thereof, comprises
a heavy chain variable (V_{H}) region that comprises complementary determining region (CDR) V_{H}CDR1, V_{H}CDR2, and V_{H}CDR3 sequences of SEQ ID NOs: 6-8, respectively; and
a light chain variable (V_{L}) region that comprises V_{L}CDR1, V_{L}CDR2, and V_{L}CDR3 sequences of SEQ ID NOs: 9-11, respectively,
and variants thereof which specifically bind to the VP1 protein.

In some embodiments:
the V_{H} region comprises a sequence at least 80, 85, 90, 95, 97, 98, 99, or 100% identical to SEQ ID NO: 12, optionally wherein the V_{H} sequence has 1, 2, 3, 4, 5, or 6 alterations in the framework regions, optionally selected from one or more of V5Q, G9P, T10G, N30S, N30K, and N30Q; and
the V_{L} region comprises sequence at least 80, 85, 90, 95, 97, 98, 99, or 100% identical to SEQ ID NO: 13, optionally wherein the V_{L} sequence has 1, 2, 3, 4, 5, or 6 alterations in the framework regions.

In some embodiments, V_{H} region comprises, consists, or consists essentially of SEQ ID NO: 12 and the V_{L} region comprises, consists, or consists essentially of SEQ ID NO: 13.

In some embodiments the dosing regimen comprises identifying the BK virus genotype or the JC virus in subject. In some embodiments, the polyomavirus infection comprises a BK virus selected from one or more of genotype I, II, III, and IV, or wherein the polyomavirus infection comprises a JC virus.

In some embodiments, the subject is immuno-compromised. In some embodiments, the subject is about to undergo, is undergoing, or has undergone a transplant procedure, optionally an organ transplant or cell-based transplant procedure. In some embodiments, the transplant procedure is selected from a kidney transplant and a hematopoietic cell transplant (HCT). In some embodiments, the subject has or is at risk for having a condition selected from BK virus-associated nephropathy, BK virus-associated hemorrhagic cystitis, and JC virus-associated progressive multifocal leukoencephalopathy.

In some embodiments, the dosage in (a) is about 1 to about 100 mg/kg, or about 10-30 mg/kg, or about 1, 2, 3, 4, 5, 6, 7, 8, 9, 10, 15, 20, 25, 30, 35, 40, 45, 50, 60, 70, 80, 90, or 100 mg/kg, optionally wherein the dosage is administered intravenously or subcutaneously. In some embodiments, the further dosage in (c) is the same as or different than the dosage in (a), optionally about 1 to about 100 mg/kg, or about 10-30 mg/kg, or about 1, 2, 3, 4, 5, 6, 7, 8, 9, 10, 15, 20, 25, 30, 35, 40, 45, 50, 60, 70, 80, 90, or 100 mg/kg, optionally wherein the dosage is administered intravenously or subcutaneously.

In some embodiments, the polyomavirus infection comprises a BK virus genotype I and wherein:
the tissue concentration, optionally renal tissue concentration, at the C_{trough} ranges from about 2618 to about 3775 to about 13,061-fold above the EC₅₀ of the antibody, or antigen-binding fragment thereof; or
the tissue concentration, optionally bladder tissue concentration, at the C_{trough} ranges from about 500 to about 692 to about 1000-fold above the EC₅₀ of the antibody, or antigen-binding fragment thereof,
wherein the EC₅₀ is about 0.009±0.010 μg/mL.

In some embodiments, the polyomavirus infection comprises a BK virus genotype II and wherein:

the tissue concentration, optionally renal tissue concentration, at C_{trough} ranges from about 589 to about 849 to about 2942-fold (for example, about 600, 700, 800, 900, 1000, 1500, 2000, 2500, 3000-fold) above the EC₅₀ of the antibody, or antigen-binding fragment thereof; or
the tissue concentration, optionally bladder tissue concentration, at C_{trough} ranges from about 100 to about 156 to about 200-fold above the EC₅₀ of the antibody, or antigen-binding fragment thereof,
wherein the EC₅₀ is about 0.040±0.025 μg/mL.

In some embodiments, the polyomavirus infection comprises a BK virus genotype III and wherein:
the tissue concentration, optionally renal tissue concentration, at the C_{trough} ranges from about 253 to about 365 to about 1265-fold above the EC₅₀ of the antibody, or antigen-binding fragment thereof; or
the tissue concentration, optionally bladder tissue concentration, at the C_{trough} ranges from about 50 to about 67 to about 100-fold (for example, about 50, 55, 60, 65, 70, 75, 80, 85, 90, 95, or 100-fold) above the EC₅₀ of the antibody, or antigen-binding fragment thereof,
wherein the EC₅₀ is about 0.093±0.057 μg/mL.

In some embodiments, the polyomavirus infection comprises a BK virus genotype IV and wherein:
the tissue concentration, optionally renal tissue concentration, at the C_{trough} ranges from about 1122 to about 1618 to about 5604-fold above the EC₅₀ of the antibody, or antigen-binding fragment thereof; or
the tissue concentration, optionally bladder tissue concentration, at the C_{trough} ranges from about 100 to about 297 to about 500-fold above the EC₅₀ of the antibody, or antigen-binding fragment thereof,
wherein the EC₅₀ is about 0.021±0.020 μg/mL.

In some embodiments, the polyomavirus infection comprises a JC virus and the tissue concentration at the C_{trough} is at least about 29 to about 110 to about 158 to about 547-fold above the EC₅₀ of the antibody, or antigen-binding fragment thereof, wherein the EC₅₀ is about 0.215±0.130 μg/mL.

In some embodiments, time between (a) and (b) is about, at least about, or no more than about 1, 2, 3, 4, 5, 6, 7, 8, 9, 10, 11, 12, 13, 14, 14, 16, 17, 18, 19, 20, 21, 22, 23, 24, 25, 26, 27, 28, 29, or 30 days, or 1, 2, 3, 4, 5, 6, 7, or 8 weeks, or 1, 2, or 3 months. Certain dosing regimens comprise (b) measuring the serum or tissue levels in the subject about once every 3, 4, 5, 6, 7, 8, 9, 10, 11, 12, 13, 14, 14, 16, 17, 18, 19, 20, 21, 22, 23, 24, 25, 26, 27, 28, 29, or 30 days, or about 1-3 times every 1, 2, 3, 4, 5, 6, 7, or 8 weeks, or about 1-6 times every 1, 2, or 3 months. In some embodiments, the time between (a) and (c) is about, at least about, or no more than about 1, 2, 3, 4, 5, 6, 7, 8, 9, 10, 11, 12, 13, 14, 14, 16, 17, 18, 19, 20, 21, 22, 23, 24, 25, 26, 27, 28, 29, or 30 days, or 1, 2, 3, 4, 5, 6, 7, or 8 weeks, or 1, 2, or 3 months. In some embodiments, at least 1, 2, 3, 4, 5, 6, 7, 8, 9, 10, 11, 12, 13, 14, 15, 16, 17, 18, 19, or 20% or more of the serum, circulating concentration of the antibody, or antigen-binding fragment thereof, penetrates an infected organ. In some embodiments, the infected organ is a bladder or a kidney or the central nervous system (CNS).

In some embodiments, the mean clearance of the antibody, or antigen binding fragment thereof, is about 0.0760-0.0996 mL/day/kg. In some embodiments, the mean volume of distribution of the antibody, or antigen binding fragment thereof, is about 49.8-81.9 mL/kg.

Also included are methods for treating a BK or JC polyomavirus infection in a human subject in need thereof, comprising parenterally administering to the subject a dosage of an antibody, or an antigen-binding fragment thereof, which specifically binds to a VP1 protein of the polyomavirus, wherein the dosage is about 10-30 mg/kg, or about 10, 11, 12, 13, 14, 15, 16, 17, 18, 19, 20, 21, 22, 23, 24, 25, 26, 27, 28, 29, or 30 mg/kg.

In some embodiments, the antibody, or antigen-binding fragment thereof, comprises
a heavy chain variable (V_{H}) that comprises complementary determining region (CDR) V_{H}CDR1, V_{H}CDR2, and V_{H}CDR3 sequences of SEQ ID NOs: 6-8, respectively; and
a light chain variable (V_{L}) region that comprises V_{L}CDR1, V_{L}CDR2, and V_{L}CDR3 sequences of SEQ ID NOs: 9-11, respectively,
and variants thereof which specifically bind to the VP1 protein.

In some embodiments:
the V_{H} region comprises a sequence at least 80, 85, 90, 95, 97, 98, 99, or 100% identical to SEQ ID NO: 12, optionally wherein the V_{H} sequence has 1, 2, 3, 4, 5, or 6 alterations in the framework regions, optionally selected from one or more of V5Q, G9P, T10G, N30S, N30K, and N30Q; and
the V_{L} region comprises sequence at least 80, 85, 90, 95, 97, 98, 99, or 100% identical to SEQ ID NO: 13, optionally wherein the V_{L} sequence has 1, 2, 3, 4, 5, or 6 alterations in the framework regions.

In some embodiments, the V_{H} region comprises, consists, or consists essentially of SEQ ID NO: 12 and the V_{L} region comprises, consists, or consists essentially of SEQ ID NO: 13.

In some embodiments, the polyomavirus infection comprises a BK virus selected from one or more of genotype I, II, III, and IV. In some embodiments, the polyomavirus infection comprises a JC virus.

In some embodiments, the subject is immuno-compromised. In some embodiments, the subject is about to undergo, is undergoing, or has undergone a transplant procedure, optionally an organ transplant or cell-based transplant procedure. In some embodiments, the transplant procedure is selected from a kidney transplant and a hematopoietic cell transplant (HCT). In some embodiments, the subject has or is at risk for having a condition selected from BK virus-associated nephropathy, BK virus-associated hemorrhagic cystitis, and JC virus-associated progressive multifocal leukoencephalopathy.

In some embodiments, at least 1, 2, 3, 4, 5, 6, 7, 8, 9, 10, 11, 12, 13, 14, 15, 16, 17, 18, 19, or 20% or more of the serum, circulating concentration of the antibody, or antigen-binding fragment thereof, penetrates an infected organ. In some embodiments, the infected organ is a bladder or a kidney. In some embodiments, the mean clearance of the antibody, or antigen binding fragment thereof, is about 0.0760-0.0996 mL/day/kg. In some embodiments, the mean volume of distribution of the antibody, or antigen binding fragment thereof, is about 49.8-81.9 mL/kg. In some embodiments, the 10-30 mg/kg dosage provides optimal neutralizing activity (IC₅₀) by the subject's serum against the BK or JC polyomavirus, as measured in an in vitro or ex vivo viral assay.

In some embodiments, the dosing regimen or method reduces BK or JC viremia and/or viruria in the subject, optionally by about or at least about 10%, 20%, 30%, 40%, 50%, 60%, 70%, 80%, 90%, 100%, 200%, 300%, 400%, 500%, 1000%, 2000%, 3000%, 4000%, or 5000% or more, relative to a control or prior to treatment with the antibody, or antigen-binding fragment thereof.

In some embodiments, the dosing regimen or method reduces or improves one or more BK or JC virus-related symptoms in the subject, optionally one or more symptoms selected from blurred vision or other vision changes, brown or red urine, pain while urinating, reduced kidney function, difficulty urinating, cough, colds, trouble breathing, fever, muscle pain, muscle weakness, and/or seizures, optionally narrowed ureters, or kidney inflammation such as interstitial nephritis. In some embodiments, the dosing regimen or method comprises administering the antibody, or antigen-binding fragment thereof, in a pharmaceutical composition that comprises histidine, a saccharide optionally sucrose, and a polyol optionally a polysorbate.

Also included are pharmaceutical compositions, comprising:
an antibody, or antigen-binding fragment thereof, which is formulated for parenteral administration at a dosage of about 10-30 mg/kg, and which comprises a heavy chain variable (V_{H}) that comprises complementary determining region (CDR) V_{H}CDR1, V_{H}CDR2, and V_{H}CDR3 sequences of SEQ ID NOs: 6-8, respectively, and a light chain variable (V_{L}) region that comprises V_{L}CDR1, V_{L}CDR2, and V_{L}CDR3 sequences of SEQ ID NOs: 9-11, respectively; and
a pharmaceutically-acceptable carrier that comprises histidine, a saccharide optionally sucrose, and a polyol optionally a polysorbate.

In some embodiments, the V_{H} region comprises a sequence at least 80, 85, 90, 95, 97, 98, 99, or 100% identical to SEQ ID NO: 12, optionally wherein the V_{H} sequence has 1, 2, 3, 4, 5, or 6 alterations in the framework regions, optionally selected from one or more of V5Q, G9P, T10G, N30S, N30K, and N30Q; and
the V_{L} region comprises sequence at least 80, 85, 90, 95, 97, 98, 99, or 100% identical to SEQ ID NO: 13, optionally wherein the V_{L} sequence has 1, 2, 3, 4, 5, or 6 alterations in the framework regions.

In some embodiments, the antibody, or antigen-binding fragment thereof, is formulated for parenteral administration at a dosage of about 10, 11, 12, 13, 14, 15, 16, 17, 18, 19, 20, 21, 22, 23, 24, 25, 26, 27, 28, 29, or 30 mg/kg. Certain compositions are formulated for intravenous administration. Some compositions are formulated for subcutaneous administration.

Some embodiments include a pharmaceutical composition described herein, for use in treating a BK or JC polyomavirus infection in a human subject in need thereof. In some embodiments, the polyomavirus infection comprises a BK virus selected from one or more of genotype I, II, III, and IV. In some embodiments, the polyomavirus infection comprises a JC virus. In some embodiments, the subject is immuno-compromised. In some embodiments, the subject is about to undergo, is undergoing, or has undergone a transplant procedure, optionally an organ transplant or cell-based transplant procedure. In some embodiments, the transplant procedure is selected from a kidney transplant and a hematopoietic cell transplant (HCT). In some embodiments, the subject has or is at risk for having a condition selected from BK virus-associated nephropathy, BK virus-associated hemorrhagic cystitis, and JC virus-associated progressive multifocal leukoencephalopathy.

### BRIEF DESCRIPTION OF THE FIGURES

FIG. 1 shows the time course of exposure to MAU868 following intravenous or subcutaneous administration.

FIG. 2 shows the ex vivo serum neutralizing activity of MAU868 from patient serum relative its serum concentration. BKVEC₅₀=volume of serum required to neutralize in vitro infection by 50%.

### DETAILED DESCRIPTION

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” includes “one element”, “one or more elements” and/or “at least one element”.

By “about” is meant a quantity, level, value, number, frequency, percentage, dimension, size, amount, weight or length that varies by as much as 10, 9, 8, 7, 6, 5, 4, 3, 2 or 1% to a reference quantity, level, value, number, frequency, percentage, dimension, size, amount, weight or length.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes. As used herein, the term “antigen” includes substances that are capable, under appropriate conditions, of inducing an immune response to the substance and of reacting with the products of the immune response. For example, an antigen can be recognized by antibodies (humoral immune response) or sensitized T-lymphocytes (T helper or cell-mediated immune response), or both. Antigens can be soluble substances, such as toxins and foreign proteins, or particulates, such as bacteria and tissue cells; however, only the portion of the protein or polysaccharide molecule known as the antigenic determinant (epitopes) combines with the antibody or a specific receptor on a lymphocyte. More broadly, the term “antigen” includes any substance to which an antibody binds, or for which antibodies are desired, regardless of whether the substance is immunogenic. For such antigens, antibodies can be identified by recombinant methods, independently of any immune response.

An “antagonist” refers to biological structure or chemical agent that interferes with or otherwise reduces the physiological action of another agent or molecule. In some instances, the antagonist specifically binds to the other agent or molecule. Included are full and partial antagonists.

An “agonist” refers to biological structure or chemical agent that increases or enhances the physiological action of another agent or molecule. In some instances, the agonist specifically binds to the other agent or molecule. Included are full and partial agonists.

As used herein, the term “amino acid” is intended to mean both naturally occurring and non-naturally occurring amino acids as well as amino acid analogs and mimetics. Naturally-occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

As used herein, the term “antibody” encompasses not only intact polyclonal or monoclonal antibodies, but also fragments thereof (such as dAb, Fab, Fab′, F(ab′)2, Fv), single chain (ScFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity. Certain features and characteristics of antibodies (and antigen-binding fragments thereof) are described in greater detail herein.

An antibody or antigen-binding fragment can be of essentially any type. As is well known in the art, an antibody is an immunoglobulin molecule capable of specific binding to a target, such as an immune checkpoint molecule, through at least one epitope recognition site, located in the variable region of the immunoglobulin molecule.

The term “antigen-binding fragment” as used herein refers to a polypeptide fragment that contains at least one CDR of an immunoglobulin heavy and/or light chain that binds to the antigen of interest. In this regard, an antigen-binding fragment of the herein described antibodies may comprise 1, 2, 3, 4, 5, or all 6 CDRs of a V_{H} and V_{L} sequence from antibodies that bind to a target molecule.

The binding properties of antibodies and antigen-binding fragments thereof can be quantified using methods well known in the art (see Davies et al., Annual Rev. Biochem. 59:439-473, 1990). In some embodiments, an antibody or antigen-binding fragment thereof specifically binds to a target molecule, for example, a VP1 protein or an epitope or complex thereof, with an equilibrium dissociation constant that is about or ranges from about ≤10⁻⁷ M to about 10⁻⁸ M. In some embodiments, the equilibrium dissociation constant is about or ranges from about ≤10⁻⁹ M to about ≤10⁻¹⁰ M. In certain illustrative embodiments, an antibody or antigen-binding fragment thereof has an affinity (Kd or EC₅₀) for a target molecule (to which it specifically binds) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

A molecule such as an antibody is said to exhibit “specific binding” or “preferential binding” if it reacts or associates more frequently, more rapidly, with greater duration and/or with greater affinity with a particular cell, substance, or particular epitope than it does with alternative cells or substances, or epitopes. An antibody “specifically binds” or “preferentially binds” to a target molecule or epitope if it binds with greater affinity, avidity, more readily, and/or with greater duration than it binds to other substances or epitopes, for example, by a statistically significant amount. Typically, one member of the pair of molecules that exhibit specific binding has an area on its surface, or a cavity, which specifically binds to and is therefore complementary to a particular spatial and/or polar organization of the other member of the pair of molecules. Thus, the members of the pair have the property of binding specifically to each other. For instance, an antibody that specifically or preferentially binds to a specific epitope is an antibody that binds that specific epitope with greater affinity, avidity, more readily, and/or with greater duration than it binds to other epitopes. It is also understood by reading this definition that, for example, an antibody (or moiety or epitope) that specifically or preferentially binds to a first target may or may not specifically or preferentially bind to a second target. The term is also applicable where, for example, an antibody is specific for a particular epitope which is carried by a number of antigens, in which case the specific binding member carrying the antigen-binding fragment or domain will be able to bind to the various antigens carrying the epitope; for example, it may be cross reactive to a number of different forms of a target antigen from multiple species that share a common epitope

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kon) and the “off rate constant” (Koff) can be determined by calculation of the concentrations and the actual rates of association and dissociation. The ratio of Koff/Kon enables cancellation of all parameters not related to affinity, and is thus equal to the dissociation constant Kd. As used herein, the term “affinity” includes the equilibrium constant for the reversible binding of two agents and is expressed as Kd or EC₅₀. Affinity of a binding protein to a ligand such as affinity of an antibody for an epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the resistance of a complex of two or more agents to dissociation after dilution. In some embodiments, affinity is expressed in the terms of the half maximal effective concentration (EC₅₀), which refers to the concentration of an agent, such as an antibody, as disclosed herein, which induces a response halfway between the baseline and maximum after a specified exposure time. The EC₅₀ is commonly used as a measure of an antibody's potency.

Antibodies may be prepared by any of a variety of techniques known to those of ordinary skill in the art. See, e.g., Harlow and Lane, Antibodies: A Laboratory Manual, Cold Spring Harbor Laboratory, 1988. Monoclonal antibodies specific for a polypeptide of interest may be prepared, for example, using the technique of Kohler and Milstein, Eur. J. Immunol. 6:511-519, 1976, and improvements thereto. Also included are methods that utilize transgenic animals such as mice to express human antibodies. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995. Particular examples include the VELOCIMMUNE® platform by REGENEREX® (see, e.g., U.S. Pat. No. 6,596,541).

Antibodies can also be generated or identified by the use of phage display or yeast display libraries (see, e.g., U.S. Pat. No. 7,244,592; Chao et al., Nature Protocols. 1:755-768, 2006). Non-limiting examples of available libraries include cloned or synthetic libraries, such as the Human Combinatorial Antibody Library (HuCAL), in which the structural diversity of the human antibody repertoire is represented by seven heavy chain and seven light chain variable region genes. The combination of these genes gives rise to 49 frameworks in the master library. By superimposing highly variable genetic cassettes (CDRs=complementarity determining regions) on these frameworks, the vast human antibody repertoire can be reproduced. Also included are human libraries designed with human-donor-sourced fragments encoding a light-chain variable region, a heavy-chain CDR-3, synthetic DNA encoding diversity in heavy-chain CDR-1, and synthetic DNA encoding diversity in heavy-chain CDR-2. Other libraries suitable for use will be apparent to persons skilled in the art.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

The structures and locations of immunoglobulin variable domains may be determined by reference to Kabat, E. A. et al., Sequences of Proteins of Immunological Interest. 4th Edition. US Department of Health and Human Services. 1987, and updates thereof.

Also include are “monoclonal” antibodies, which refer to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (ScFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals). The term includes whole immunoglobulins as well as the fragments etc. described above under the definition of “antibody.”

The proteolytic enzyme papain preferentially cleaves IgG molecules to yield several fragments, two of which (the F (ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH::VL heterodimer including an antigen-binding site which retains much of the antigen recognition and binding capabilities of the native antibody molecule. See Inbar et al., PNAS USA. 69:2659-2662, 1972; Hochman et al., Biochem. 15:2706-2710, 1976; and Ehrlich et al., Biochem. 19:4091-4096, 1980.

In certain embodiments, single chain Fv (scFV) antibodies are contemplated. For example, Kappa bodies (Ill et al., Prot. Eng. 10:949-57, 1997); minibodies (Martin et al., EMBO J 13:5305-9, 1994); diabodies (Holliger et al., PNAS 90:6444-8, 1993); or Janusins (Traunecker et al., EMBO J 10:3655-59, 1991; and Traunecker et al., Int. J. Cancer Suppl. 7:51-52, 1992), may be prepared using standard molecular biology techniques following the teachings of the present application with regard to selecting antibodies having the desired specificity.

A single chain Fv (scFv) polypeptide is a covalently linked V_{H}::V_{L} heterodimer which is expressed from a gene fusion including V_{H}- and V_{L}-encoding genes linked by a peptide-encoding linker. Huston et al. (PNAS USA. 85 (16): 5879-5883, 1988). A number of methods have been described to discern chemical structures for converting the naturally aggregated—but chemically separated—light and heavy polypeptide chains from an antibody V region into an scFv molecule which will fold into a three-dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, the antibodies or antigen-binding fragments thereof are humanized. These embodiments refer to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio et al., PNAS USA 86:4220-4224, 1989; Queen et al., PNAS USA. 86:10029-10033, 1988; Riechmann et al., Nature. 332:323-327, 1988). Illustrative methods for humanization of antibodies include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato et al., Cancer Res. 53:851-856, 1993; Riechmann et al., Nature 332:323-327, 1988; Verhoeyen et al., Science 239:1534-1536, 1988; Kettleborough et al., Protein Engineering. 4:773-3783, 1991; Maeda et al., Human Antibodies Hybridoma 2:124-134, 1991; Gorman et al., PNAS USA. 88:4181-4185, 1991; Tempest et al., Bio/Technology 9:266-271, 1991; Co et al., PNAS USA. 88:2869-2873, 1991; Carter et al., PNAS USA. 89:4285-4289, 1992; and Co et al., J Immunol. 148:1149-1154, 1992. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies are “chimeric” antibodies. In this regard, a chimeric antibody is comprised of an antigen-binding fragment of an antibody operably linked or otherwise fused to a heterologous Fc portion of a different antibody. In certain embodiments, the Fc domain or heterologous Fc domain is of human origin. In certain embodiments, the Fc domain or heterologous Fc domain is of mouse origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

As used herein, a subject “at risk” of developing a disease, or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

“Biocompatible” refers to materials or compounds which are generally not injurious to biological functions of a cell or subject and which will not result in any degree of unacceptable toxicity, including allergenic and disease states.

The term “binding” refers to a direct association between two molecules, due to, for example, covalent, electrostatic, hydrophobic, and ionic and/or hydrogen-bond interactions, including interactions such as salt bridges and water bridges.

The term “bioavailability” refers to the systemic availability (e.g., blood/plasma levels) of a given amount of agent (e.g., antibody) administered to a patient. Bioavailability is an absolute term that indicates measurement of both the time (rate) and total amount (extent) of agent that reaches the general circulation from an administered dosage form.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “effector function” in the context of antibodies refers to the ability of that antibody to engage with other arms of the immune system, including for example, the activation of the classical complement pathway, or through engagement of Fc receptors. Complement dependent pathways are primarily driven by the interaction of C1q with the C1 complex with clustered antibody Fc domains. Antibody dependent cellular cytotoxicity (ADCC), is primarily driven by the interaction of Fc receptors (FcRs) on the surface of effector cells (natural killer cells, macrophages, monocytes and eosinophils) which bind to the Fc region of an IgG which itself is bound to a target cell. Fc receptors (FcRs) are key immune regulatory receptors connecting the antibody mediated (humoral) immune response to cellular effector functions. Receptors for all classes of immunoglobulins have been identified, including FcγR (IgG), FcεRI (IgE), FcαRI (IgA), FcμR (IgM) and FcδR (IgD). There are at least three classes of receptors for human IgG found on leukocytes: CD64 (FcγRI), CD32 (FcγRIIa, FcγRIIb and FcγRIIc) and CD16 (FcγRIIIa and FcγRIIIb). FcγRI is classed as a high affinity receptor (nanomolar range KD) while FcγRII and FcγRIII are low to intermediate affinity (micromolar range KD). Upon Fc binding a signaling pathway is triggered which results in the secretion of various substances, such as lytic enzymes, perforin, granzymes and tumour necrosis factor, which mediate in the destruction of the target cell. The level of ADCC effector function various for human IgG subtypes. Although this is dependent on the allotype and specific FcvR, in simple terms ADCC effector function is “high” for human IgG1 and IgG3, and “low” for IgG2 and IgG4.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin.

Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

An “epitope” includes that portion of an antigen or other macromolecule capable of forming a binding interaction with the variable region binding pocket or paratope of an antibody. Such binding interaction can be manifested as an intermolecular contact with one or more amino acid residues of a CDR. An epitope can be a linear peptide sequence or can be composed of noncontiguous amino acid sequences (i.e., “conformational” or “discontinuous”). Thus, epitopes can be contiguous or non-contiguous in relation to the primary structure of the antigen, for example, a VP1 polypeptide. A binding protein can recognize one or more amino acid sequences; therefore, an epitope can define more than one distinct amino acid sequence. In particular embodiments, an epitope comprises, consists, or consists essentially of about, at least about, or no more than about 3, 4, 5, 6, 7, 8, 9, 10, 11, 12, 13, 14, 15, 16, 17, 18, 19, or 20 contiguous amino acids (i.e., a linear epitope) or non-contiguous amino acids (i.e., conformational epitope) of a reference sequence or target molecule described herein (see, e.g., Table V1). Epitopes recognized by binding protein can be determined by peptide mapping and sequence analysis techniques well known to one of skill in the art.

The term “half maximal effective concentration” or “EC₅₀” refers to the concentration of an agent (e.g., antibody) as described herein at which it induces a response halfway between the baseline and maximum after some specified exposure time; the EC₅₀ of a graded dose response curve therefore represents the concentration of a compound at which 50% of its maximal effect is observed. EC50 also represents the plasma concentration required for obtaining 50% of a maximum effect in vivo. For example, in some instances, the EC₅₀ is the concentration of antibody at which virus infection is neutralized by 50%. Similarly, the “EC₉₀” refers to the concentration of an agent or composition at which 90% of its maximal effect is observed. For example, in some instances, the EC₉₀ is the concentration of antibody at which virus infection is neutralized by 90%. The “EC₉₀” can be calculated from the “EC₅₀” and the Hill slope, or it can be determined from the data directly, using routine knowledge in the art. In some embodiments, the EC₅₀ of an agent (e.g., antibody) is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC₅₀ value of about 1 nM or less.

The term “half-maximal inhibitory concentration” or “IC₅₀” refers to the concentration of an agent (e.g., antibody) described herein which induces a signal halfway (50%) between the baseline control and the maximum possible signal. For example, in some instances, the IC₅₀ is the concentration of antibody at which 50% of the available binding sites on the VP1 antigen are occupied.

The “half-life” of an agent such as an antibody can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 1.5, 2, 3, 4, 5, 6, 7, 8, 9, 10, 15, 20, 30, 40, 50, 60, 70, 80, 90, 100 or more times (e.g., 500, 1000 times) (including all integers and ranges in between e.g., 1.5, 1.6, 1.7, 1.8, etc.) the amount produced by no composition (e.g., the absence of agent) or a control composition. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) in the amount produced by no composition (e.g., the absence of an agent) or a control composition. Examples of comparisons and “statistically significant” amounts are described herein.

“Neutralization” refers to the inhibition of viral infection of a host cell, as demonstrated by the absence of viral gene expression. Without being held to any one theory, mechanisms of neutralization by a particular antibody could include blocking the interaction of viral capsid proteins with cell surface receptors or disruption of any stage of the entry and trafficking process prior to delivery of the viral genome to the nucleus of the host cell.

The terms “polypeptide,” “protein” and “peptide” are used interchangeably and mean a polymer of amino acids not limited to any particular length. The term “enzyme” includes polypeptide or protein catalysts. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “isolated” polypeptide or protein referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or non-covalent interaction) with portions of a protein with which the “isolated protein” is associated in nature, (6) is operably associated (by covalent or non-covalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise). The term “isolated antibody” includes an antibody that is substantially free of other antibodies having different antigenic specificities. An isolated antibody that specifically binds to one antigen may, however, have cross-reactivity to other antigens. Moreover, an isolated antibody may be substantially free of other cellular material and/or chemicals

The term “polynucleotide” and “nucleic acid” includes mRNA, RNA, CRNA, cDNA, and DNA. The term typically refers to polymeric form of nucleotides of at least 10 bases in length, either ribonucleotides or deoxynucleotides or a modified form of either type of nucleotide. The term includes single and double stranded forms of DNA. The terms “isolated DNA” and “isolated polynucleotide” and “isolated nucleic acid” refer to a molecule that has been isolated free of total genomic DNA of a particular species. Therefore, an isolated DNA segment encoding a polypeptide refers to a DNA segment that contains one or more coding sequences yet is substantially isolated away from, or purified free from, total genomic DNA of the species from which the DNA segment is obtained.

In certain embodiments, the “purity” of any given agent (e.g., an antibody) in a composition may be defined. For instance, certain compositions may comprise an agent such as a polypeptide agent that is at least 80%, 85%, 90%, 91%, 92%, 93%, 94%, 95%, 96%, 97%, 98%, 99%, or 100% pure on a protein basis or a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

Certain embodiments include biologically active “variants” and “fragments” of the polypeptides (e.g., antibodies) described herein, and the polynucleotides that encode the same. “Variants” contain one or more substitutions, additions, deletions, and/or insertions relative to a reference polypeptide or polynucleotide (see, e.g., the Tables and the Sequence Listing). A variant polypeptide or polynucleotide comprises an amino acid or nucleotide sequence with at least about 50%, 55%, 60%, 65%, 70%, 75%, 80%, 85%, 90%, 91%, 92%, 93%, 94%, 95%, 96%, 97%, 98%, 99% or more sequence identity or similarity or homology to a reference sequence, as described herein, and substantially retains the activity of that reference sequence. Also included are sequences that consist of or differ from a reference sequences by the addition, deletion, insertion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150 or more amino acids or nucleotides and which substantially retain the activity of that reference sequence. In certain embodiments, the additions or deletions include C-terminal and/or N-terminal additions and/or deletions.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

The term “solubility” refers to the property of an agent (e.g., antibody) provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaPO₄). In specific embodiments, solubility is measured at relatively lower pH (e.g., pH 6.0) and relatively higher salt (e.g., 500 mM NaCl and 10 mM NaPO₄). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” or a “patient” or a “patient in need thereof” includes a mammalian subject such as a human subject.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Therapeutic response” refers to improvement of symptoms (whether or not sustained) based on administration of one or more therapeutic agents.

As used herein, the terms “therapeutically effective amount”, “therapeutic dose,” “prophylactically effective amount,” or “diagnostically effective amount” is the amount of an agent (e.g., anti-polyomavirus antibody) needed to elicit the desired biological response following administration.

As used herein, “treatment” of a subject (e.g., a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product that is most frequently observed in a population and is thus arbitrarily designed the “normal” or “wild-type” form of the gene.

Each embodiment in this specification is to be applied to every other embodiment unless expressly stated otherwise.

As noted above, embodiments of the present disclosure relate to dosing regimens, and optimal dosages, of one or more polyomavirus neutralizing antibodies, including antigen-binding fragments thereof.

For example, certain embodiments relate to a dosing regimen for treatment of a BK or JC polyomavirus infection in a human subject in need thereof, comprising
(a) parenterally administering to the subject a dosage of a polyomavirus neutralizing antibody, or an antigen-binding fragment thereof, that specifically binds to a VP1 protein of the polyomavirus;
(b) measuring serum or tissue levels of the antibody, or antigen-binding fragment thereof, in the subject; and
(c) administering a further dosage of the antibody, or antigen-binding fragment thereof, before the serum or tissue trough concentration (C_{trough}) falls below about 3-860 μg/mL,
wherein the dosing regimen maintains the serum or tissue concentration of the antibody, or antigen-binding fragment thereof, above the C_{trough} throughout the treatment.

The “trough concentration” or “C_{trough}” refers to lowest serum or tissue concentration of an agent before administration of the next dosage. For periodic administrations, the C_{trough} is typically measured just before the administration of the next dosage, for example, to maintain optimal virus neutralizing activity and avoid overdosing.

In certain embodiments, the C_{trough} in step (c) is determined for plasma, and the dosing regimen comprises administering the further dosage before the plasma C_{trough} falls below about 150-860 μg/mL, or about 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 20, 630, 640, 650, 660, 670, 680, 690, 600, 710, 720, 730, 740, 750, 760, 770, 780, 790, 800, 810, 820, 830, 840, 850, or 860 μg/mL.

In some embodiments, the C_{trough} in step (c) is for a particular tissue, for instance, renal tissue or bladder tissue. Merely for illustrative purposes, the plasma concentration of the antibody, or antigen-binding fragment thereof, can be used to determine the renal tissue concentration (e.g., renal C_{trough} is about 13.7% of plasma C_{trough}) or the bladder tissue concentration (e.g., bladder C_{trough} is about 1% of plasma C_{trough}).

In some embodiments, the C_{trough} in step (c) is determined for renal tissue, and the dosing regimen comprises administering the further dosage before the C_{trough} in renal tissue falls below about 23.5-120 μg/mL, or about 23.5, 25, 30, 35, 40, 45, 50, 55, 60, 65, 70, 75, 80, 85, 90, 95, 100, 105, 110, 115, or 120, μg/mL. In certain of these and related embodiments, the subject has or is at risk for having BK virus-associated nephropathy.

In some embodiments, the C_{trough} in step (c) is determined for bladder tissue, and the dosing regimen comprises administering the further dosage before the C_{trough} in bladder tissue falls below about 3-10 μg/mL, or about 3, 3.5, 4, 4.5, 5, 5.5, 6, 6.5, 7, 7.5, 8, 8.5, 9, 9.5 or 10 μg/mL. In certain of these and related embodiments, the subject has or is at risk for having BK virus-associated hemorrhagic cystitis.

Some embodiments, either practiced separately or as part of a dosing regimen described herein, relate to optimal dosages of one or more polyomavirus neutralizing antibodies, including antigen-binding fragments thereof. For instance, certain embodiments relate to methods for treating a BK or JC polyomavirus infection in a human subject in need thereof, comprising parenterally administering to the subject a dosage of a polyomavirus neutralizing antibody, or an antigen-binding fragment thereof, that specifically binds to a VP1 protein of the polyomavirus, wherein the dosage is about 10-30 mg/kg, for example, about 10, 11, 12, 13, 14, 15, 16, 17, 18, 19, 20, 21, 22, 23, 24, 25, 26, 27, 28, 29, or 30 mg/kg, or about 10-15, 10-20, 10-25, 15-20, 15-25, 15-30, 20-30, or 25-30 mg/kg.

The terms “BKV” or “BK virus” refer to a member of the family Polyomaviridae, genus Orthopolyomavirus. Polyomaviruses are icosahedral, non-enveloped, double-stranded DNA viruses with a genome of approximately 5,000 base pairs. They measure approximately 40-45 nM in diameter (Bennett et al., Microbes and Infection. 14:672-683, 2012). “JCV” or “JC virus” refers to a member of the family Polyomaviridae, genus Orthopolyomavirus. JCV is related to BKV, and is also an icosahedral, non-enveloped, double-stranded DNA virus with a genome of approximately 5,000 base pairs. They measure approximately 40-45 nM in diameter (Johne et al., Arch. Virol. 156:1627-1634, 2011).

As noted above, an antibody, or an antigen-binding fragment thereof, specifically binds to a VP1 protein of the polyomavirus. The term “VP1” refers to the major polyoma virus capsid subunit protein. “VP1 pentamers” are composed of five monomers of VP1. Exemplary VP1 proteins are provided in Table V1 below.

TABLE VI
VP1 Protein Sequences

| | | SEQ ID |
|---|---|---|
| Description | Sequence | NO: |
| VP1 BKV | MAPTKRKGECPGAAPKKPKEPVQVPKLLIKGGVEVLEVKTGVDAITEVECF | 1 |
| genotype I | LNPEMGDPDENLRGFSLKLSAENDFSSDSPERKMLPCYSTARIPLPNLNED | |
| | LTCGNLLMWEAVTVQTEVIGITSMLNLHAGSQKVHEHGGGKPIQGSNFHFF | |
| | AVGGDPLEMQGVLMNYRTKYPEGTITPKNPTAQSQVMNTDHKAYLDKNNAY | |
| | PVECWIPDPSRNENTRYFGTFTGGENVPPVLHVTNTATTVLLDEQGVGPLC | |
| | KADSLYVSAADICGLFTNSSGTQQWRGLARYFKIRLRKRSVKNPYPISFLL | |
| | SDLINRRTQRVDGQPMYGMESQVEEVRVFDGTERLPGDPDMIRYIDKQGQL | |
| | QTKML | |
| VP1 BKV | MAPTKRKGECPGAAPKKPKEPVQVPKLLIKGGVEVLEVKTGVDAITEVECF | 2 |
| genotype II | LNPEMGDPDDNLRGYSLKLTAENAFDSDSPDKKMLPCYSTARIPLPNLNED | |
| | LTCGNLLMWEAVTVKTEVIGITSMLNLHAGSQKVHENGGGKPVQGSNFHFF | |
| | AVGGDPLEMQGVLMNYRTKYPQGTITPKNPTAQSQVMNTDHKAYLDKNNAY | |
| | PVECWIPDPSRNENTRYFGTYTGGENVPPVLHVTNTATTVLLDEQGVGPLC | |
| | KADSLYVSAADICGLFTNSSGTQQWRGLARYFKIRLRKRSVKNPYPISFLL | |
| | SDLINRRTQKVDGQPMYGMESQVEEVRVFDGTEQLPGDPDMIRYIDRQGQL | |
| | QTKMV | |
| VP1 BKV | MAPTKRKGECPGAAPKKPKEPVQVPKLLIKGGVEVLEVKTGVDAITEVECF | 3 |
| genotype | LNPEMGDPDDHLRGYSQHLSAENAFDSDSPDKKMLPCYSTARIPLPNLNED | |
| III | LTCGNLLMWEAVTVKTEVIGITSMLNLHAGSQKVHENGGGKPVQGSNFHFF | |
| | AVGGDPLEMQGVLMNYRTKYPQGTITPKNPTAQSQVMNTDHKAYLDKNNAY | |
| | PVECWIPDPSKNENTRYFGTYTGGENVPPVLHVTNTATTVLLDEQGVGPLC | |
| | KADSLYVSAADICGLFTNSSGTQQWRGLARYFKIRLRKRSVKNPYPISFLL | |
| | SDLINRRTQKVDGQPMYGMESQVEEVRVFDGTEQLPGDPDMIRYIDRQGQL | |
| | QTKMV | |
| VP1 BKV | MAPTKRKGECPGAAPKKPKEPVQVPKLLIKGGVEVLEVKTGVDAITEVECF | 4 |
| genotype IV | LNPEMGDPDNDLRGYSLRLTAETAFDSDSPDRKMLPCYSTARIPLPNLNED | |
| | LTCGNLLMWEAVTVKTEVIGITSMLNLHAGSQKVHENGGGKPIQGSNFHFF | |
| | AVGGDPLEMQGVLMNYRTKYPEGTVTPKNPTAQSQVMNTDHKAYLDKNNAY | |
| | PVECWIPDPSRNENTRYFGTYTGGENVPPVLHVTNTATTVLLDEQGVGPLC | |
| | KADSLYVSAADICGLFTNSSGTQQWRGLPRYFKIRLRKRSVKNPYPISFLL | |
| | SDLINRRTQRVDGQPMYGMESQVEEVRVFDGTEQLPGDPDMIRYIDRQGQL | |
| | QTKMV | |
| VP1 JCV | MAPTKRKGERKDPVQVPKLLIRGGVEVLEVKTGVDSITEVECFLTPEMGDP | 5 |
| | DEHLRGFSKSISISDTFESDSPNKDMLPCYSVARIPLPNLNEDLTCGNILM | |
| | WEAVTLKTEVIGVTTLMNVHSNGQATHDNGAGKPVQGTSFHFFSVGGEALE | |
| | LQGVVFNYRTKYPDGTIFPKNATVQSQVMNTEHKAYLDKNKAYPVECWVPD | |
| | PTRNENTRYFGTLTGGENVPPVLHITNTATTVLLDEFGVGPLCKGDNLYLS | |
| | AVDVCGMFTNRSGSQQWRGLSRYFKVQLRKRRVKNPYPISFLLTDLINRRT | |
| | PRVDGQPMYGMDAQVEEVRVFEGTEELPGDPDMMRYVDRYGQLQTKML | |

In certain embodiments, an antibody, or antigen-binding fragment thereof, specifically binds to one or more VP1 proteins selected from Table V1. In particular embodiments, an antibody, or antigen-binding fragment thereof, specifically binds to, or cross-reacts with, each of the VP1 proteins set forth SEQ ID NOs: 1-5, that is, it binds to VP1 from all of BKV serotypes I-IV and JC virus. In particular embodiments, an antibody, or antigen-binding fragment thereof, neutralizes or otherwise reduces or inhibits replication of all of BKV serotypes I-IV and JC virus. In some embodiments, an antibody, or antigen-binding fragment thereof, specifically binds to a VP1 conformational epitope that comprises 1, 2, or 3 defined contact residues in VP1, for example, any one or more of Y169, R170, and K172 of VP1.

In some embodiments, an antibody or antigen-binding fragment thereof is characterized by or comprises a heavy chain variable (V_{H}) region that comprises one or more complementary determining region (CDR) sequences including V_{H}CDR1, V_{H}CDR2, and V_{H}CDR3 sequences, and a light chain variable (V_{L}) region that comprises one or more CDR sequences such as V_{L}CDR1, V_{L}CDR2, and V_{L}CDR3 sequences. Exemplary V_{H}, V_{H}CDR1, V_{H}CDR2, V_{H}CDR3, V_{L}, V_{L}CDR1, V_{L}CDR2, and V_{L}CDR3 sequences are provided in Table A1 and Table A2 below.

TABLE A1
CDR Sequences

| | | |
|---|---|---|
| | | SEQ ID |
| Description | Sequence | NO: |

MAU868

| | | |
|---|---|---|
| V_{H}CDR1 | GFTFNNYWMT | 6 |
| V_{H}CDR2 | NIKKDGSEKYYVDSVRG | 7 |
| V_{H}CDR3 | VRSGRYFALDD | 8 |
| V_{L}CDR1 | GGDNIGSRPVH | 9 |
| V_{L}CDR2 | DDSNRPS | 10 |
| V_{L}CDR3 | QVWSSSTDHP | 11 |

TABLE A2
VH and VL Sequences

| | | |
|---|---|---|
| | | SEQ ID |
| Description | Sequence | NO: |

MAU868

| | | |
|---|---|---|
| Heavy chain | QVQLVESGGTLVQPGGSLRLSCAASGFTFNNYWMTWVRQAPGKGLEWV | 12 |
| variable | ANIKKDGSEKYYVDSVRGRFTISRDNAKNSLFLQMNSLRPEDTAVYFC | |
| region (V_{H}) | ATVRSGRYFALDDWGQGTLVTVSSASTKGPSVFPLAPSSKSTSGGTAA | |
| | LGCLVKDYFPEPVTVSWNSGALTSGVHTFPAVLQSSGLYSLSSVVTVP | |
| | SSSLGTQTYICNVNHKPSNTKVDKRVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIE | |
| | KTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVMH | |
| | EALHNHYTQKSLSLSPGK | |
| Light chain | QSVLTQPPSVSVAPGKTARITCGGDNIGSRPVHWYQQKPGQAPILVVY | 13 |
| variable | DDSNRPSGIPERFSGSNSGNTATLTISRVEAGDEADYYCQVWSSSTDH | |
| region (V_{L}) | PFGGGTKVTVLGQPKAAPSVTLFPPSSEELQANKATLVCLISDFYPGA | |
| | VTVAWKADSSPVKAGVETTTPSKQSNNKYAASSYLSLTPEQWKSHRSY | |
| | SCQVTHEGSTVEKTVAPTECS | |

Thus, in certain embodiments, an antibody or antigen-binding fragment thereof comprises a (V_{H} region that comprises V_{H}CDR1, V_{H}CDR2, and V_{H}CDR3 sequences selected from Table A1 and variants thereof which specifically bind to a polyomavirus VP1 protein (selected, for example, from Table V1); and a V_{L} region that comprises V_{L}CDR1, V_{L}CDR2, and V_{L}CDR3 sequences selected from Table A1 and variants thereof which specifically bind to a polyomavirus VP1 protein (selected, for example, from Table V1). In certain embodiments, the CDR sequences are as follows: the V_{H}CDR1, V_{H}CDR2, and V_{H}CDR3 sequences comprise SEQ ID NOs: 6-8, respectively, and the V_{L}CDR1, V_{L}CDR2, and V_{L}CDR3 sequences comprise SEQ ID NOs: 9-11, respectively, including variants thereof that specifically bind to the polyomavirus VP1 protein. Included are variants that have 1, 2, 3, 4, 5, or 6 total alterations in one or more of the CDR regions, for example, one or more the V_{H}CDR1, V_{H}CDR2, V_{H}CDR3, V_{L}CDR1, V_{L}CDR2, and/or V_{L}CDR3 sequences described herein. Exemplary “alterations” include amino acid substitutions, additions, and deletions.

In certain embodiments, the V_{H} sequence is at least 80, 85, 90, 95, 97, 98, 99, or 100% identical to a sequence selected from Table A2, including, for example, wherein the V_{H} sequence has 1, 2, 3, 4, 5, or 6 alterations in one or more framework regions. In some embodiments, the V_{L} sequence is at least 80, 85, 90, 95, 97, 98, 99, or 100% identical to a sequence selected from Table A2, including, for example, wherein the V_{L} sequence has 1, 2, 3, 4, 5, or 6 alterations in one or more framework regions. Specific examples include one or more substitutions selected from V5Q, G9P, T10G, N30S, N30K, and N30Q. In some embodiments, the V_{H} and V_{L} sequences of an antibody or antigen-binding fragment are as follows: the V_{H} sequence comprises, consists, or consists essentially of SEQ ID NO: 12, and the V_{L} sequence comprises, consists, or consists essentially of SEQ ID NO: 13. In specific embodiments, the antibody is MAU868 (also P8D11).

Some dosing regimens or methods include diagnosing the subject with a polyomavirus infection, for example, by identifying BK or JC viruria, or BK or JC viremia. In some embodiments, the dosing regimen comprises the step of identifying or diagnosing the BK virus genotype or the JC virus in subject. In some embodiments, the polyomavirus infection comprises a BK virus selected from one or more of genotype I, II, III, and IV. In some embodiments, the polyomavirus infection comprises only one of BK virus genotypes I, II, III, or IV. In some embodiments, the polyomavirus infection comprises two, three, or all four of BK virus genotypes I, II, III, and/or IV. In some embodiments, the polyomavirus infection comprises a JC virus, alone or in combination with any one, two, three, or four of BK virus genotypes I, II, III, and/or IV. Methods of identifying, diagnosing, or measuring viremia, viruria, and BK virus genotypes and JC virus are known in the art, and include Real-Time PCR, high-resolution melt analysis (HRMA), and other techniques (see, for example, Luo et al., J Virol. 83:2285-2297, 2009; Gambarino et al., Mol Biotechnol. 49:151-8, 2011; Matsuda et al., J Med Virol. 83:2128-34, 2011; and Toan et al., Transplantation Proceedings. 51:2683-2688, 2019).

In some embodiments, the subject is “immunocompromised”, or has an “immunodeficiency”, which refers to a state in which the subject's immune system ability to fight infectious diseases is significantly reduced or entirely absent. In some embodiments, an immunodeficiency is secondary, or acquired, and is the result of surgery, injury, or treatment with various agents, for example, immunosuppressive drugs related to organ or cell transplants, glucocorticoids, chemotherapeutics, and disease-modifying antirheumatic drugs, or exposure to environmental toxins such as heavy metals, pesticides, or petrochemicals. For treatment related aspects, the term “immunosuppression” more generally refers to the beneficial and potential adverse effects of decreasing the function of the immune system, while the term “immunodeficiency” refers mainly to the adverse effect of increased risk for infection. In some instances, secondary immunodeficiencies are caused by specific diseases or conditions. Examples include many types of cancer, particularly those of the bone marrow and blood cells (e.g., leukemia, lymphoma, multiple myeloma), and certain chronic infections. Immunodeficiency is also the hallmark of acquired immunodeficiency syndrome (AIDS), caused by the human immunodeficiency virus (HIV). Various hormonal and metabolic disorders can also cause immunodeficiencies, including anemia, hypothyroidism, and hyperglycemia. In some instances, an immunodeficiency is related to aging, for example, wherein the subject is about or at least about 60, 65, 70, 75, 80, 85, 90, 95, or 100 years of age. In some instances, an immunodeficiency is primary, or congenital, resulting from a genetic disorder in the subject.

Examples of immunodeficiencies include humoral immunodeficiencies (including B cell deficiency or dysfunction), which are generally characterized by hypogammaglobulinemia (decrease of one or more types of antibodies) and/or agammaglobulinemia (lack of all or most antibody production); T cell deficiencies, characterized, for example, by reduced T cell counts or delayed hypersensitivity skin tests; granulocyte deficiency, including decreased numbers of granulocytes (granulocytopenia or, if absent, agranulocytosis) such as of neutrophil granulocytes (termed neutropenia), and decreased function of individual granulocytes; asplenia, characterized by lack of spleen function; and complement deficiency, characterized by reduced function of the complement system.

In some embodiments, the subject is about to undergo, is undergoing, or has undergone a transplant procedure, for example, an organ transplant or cell-based transplant procedure. Examples of organ transplants include kidney (or renal), heart, liver, lung, pancreas, intestine, thymus, and uterus transplants. Examples of cell-based transplants include hematopoietic cell transplants (HCTs), such as syngeneic, autologous, and allogeneic HCTs, among others. In particular embodiments, the transplant is an allograft transplant, that is, a transplant of an organ, tissue, or cell between two genetically non-identical members of the same species. In some embodiments, the subject has an immunodeficiency that is associated with, or caused by, immunosuppressive therapies related to the transplant procedure.

In some embodiments, the subject has one or more symptoms of a BK or JC virus infection. For instance, in some instances, a subject has any one or more of blurred vision or other vision changes, brown or red urine, pain while urinating, reduced kidney function, difficulty urinating, cough, colds, trouble breathing, fever, muscle pain, muscle weakness, and/or seizures. Such symptoms can arise, for example, from narrowed ureters, interstitial nephritis, or kidney inflammation more generally.

In particular embodiments, the subject has or is at risk for having a condition selected from BK virus-associated nephropathy, BK virus-associated hemorrhagic cystitis, and JC virus-associated progressive multifocal leukoencephalopathy (PML). The terms “BKV nephropathy” or “BK virus-associated nephropathy” or “BKVAN” refer to the inflammatory interstitial nephropathy resulting from the lytic infection with BK virus, characterized by viral cytopathogenic changes and viral gene expression, primarily in the renal tubular epithelium. “Hemorrhagic cystis” or “BK virus-associated hemorrhagic cystitis” refers to an inflammation of the bladder, typically defined by lower urinary tract symptoms that include dysuria, hematuria, and hemorrhage. “JC virus-associated PML” refers to a rare and often fatal viral disease characterized by progressive damage or inflammation of the white matter of the brain, often at multiple locations (multifocal).

As noted above, the dosing regimens described herein, including methods of treating a polyomavirus infection, comprise the step or steps of monitoring or measuring serum or tissue levels of the polyomavirus neutralizing antibody, or antigen-binding fragment thereof, in the subject, for example, to achieve a dosing regimen that maintains the serum or tissue C_{trough} of the antibody at a minimum level or within a minimum range. In some embodiments, time between the dosage administration step of (a) and the monitoring or measuring step of (b) is about, at least about, or no more than about 1, 2, 3, 4, 5, 6, 7, 8, 9, 10, 11, 12, 13, 14, 14, 16, 17, 18, 19, 20, 21, 22, 23, 24, 25, 26, 27, 28, 29, or 30 days, or 1, 2, 3, 4, 5, 6, 7, or 8 weeks, or 1, 2, or 3 months. Some embodiments comprise (b) measuring the serum or tissue levels in the subject about once every 3, 4, 5, 6, 7, 8, 9, 10, 11, 12, 13, 14, 14, 16, 17, 18, 19, 20, 21, 22, 23, 24, 25, 26, 27, 28, 29, or 30 days, or about 1,3 times every 1, 2, 3, 4, 5, 6, 7, or 8 weeks, or about 1-6 times every 1, 2, or 3 months.

Certain embodiments include administering a further dosage of the antibody, or antigen-binding fragment thereof, before the serum or tissue C_{trough} falls below a defined level relative to the than the serum or tissue EC₅₀ of the antibody, or antigen-binding fragment thereof, with respect to the polyomavirus in the subject. That is, certain of the dosing regimens and methods provided herein maintain the C_{trough} at a minimum level or within a range relative to the EC₅₀ of the polyomavirus.

For example, in some embodiments, the polyomavirus infection comprises a BK virus genotype I and the tissue concentration, for example, renal tissue concentration, at the C_{trough} ranges from about 2618 to about 3775 to about 13,061-fold (for example, about 2600, 3000, 3500, 4000, 4500, 5000, 4500, 5000, 5500, 6000, 6500, 7000, 7500, 8000, 8500, 9000, 9500, 10,000, 10,500, 11,000, 11,500, 12,000, 12,500, or 13,000-fold) above the EC₅₀ of the antibody, or antigen-binding fragment thereof, wherein the EC₅₀ is about 0.009±0.010 μg/mL. In some embodiments, the polyomavirus infection comprises a BK virus genotype I and the tissue concentration, for example, bladder tissue concentration, at the C_{trough} ranges from about 500 to about 692 to about 1000-fold (for example, about 500, 600, 700, 800, 900, or 1000-fold) above the EC₅₀ of the antibody, or antigen-binding fragment thereof, wherein the EC₅₀ is about 0.009±0.010 μg/mL.

In certain embodiments, the polyomavirus infection comprises a BK virus genotype II and the tissue concentration, for example, renal tissue concentration, at C_{trough} ranges from about 589 to about 849 to about 2942-fold (for example, about 600, 700, 800, 900, 1000, 1500, 2000, 2500, 3000-fold) above the EC₅₀ of the antibody, or antigen-binding fragment thereof, wherein the EC₅₀ is about 0.040±0.025 μg/mL. In some embodiments, the polyomavirus infection comprises a BK virus genotype II and the tissue concentration, for example, bladder tissue concentration, at C_{trough} ranges from about 100 to about 156 to about 200-fold (for example, about 110, 120, 130, 140, 150, 160, 170, 180, 190, or 200-fold) above the EC₅₀ of the antibody, or antigen-binding fragment thereof, wherein the EC₅₀ is about 0.040±0.025 μg/mL.

In some embodiments, the polyomavirus infection comprises a BK virus genotype III and the tissue concentration, for example, renal tissue concentration, at the C_{trough} ranges from about 253 to about 365 to about 1265-fold (for example, about 250, 300, 350, 400, 450, 500, 550, 600, 650, 700, 750, 800, 850, 900, 950, 1000, 1150, 1200, 1250, 1300-fold) above the EC₅₀ of the antibody, or antigen-binding fragment thereof, wherein the EC₅₀ is about 0.093±0.057 μg/mL. In some embodiments, the polyomavirus infection comprises a BK virus genotype III and the tissue concentration, for example, bladder tissue concentration, at the C_{trough} ranges from about 50 to about 67 to about 100-fold (for example, about 50, 55, 60, 65, 70, 75, 80, 85, 90, 95, or 100-fold) above the EC₅₀ of the antibody, or antigen-binding fragment thereof, wherein the EC₅₀ is about 0.093±0.057 μg/mL.

In some instances, the polyomavirus infection comprises a BK virus genotype IV and the tissue concentration, for example, renal tissue concentration, at the C_{trough} ranges from about 1122 to about 1618 to about 5604-fold (for example, about 1100, 1200, 1500, 2000, 2500, 3000, 3500, 4000, 4500, 5000, 5500, 6000-fold) above the EC₅₀ of the antibody, or antigen-binding fragment thereof, wherein the EC₅₀ is about 0.021±0.020 μg/mL. In some instances, the polyomavirus infection comprises a BK virus genotype IV and the tissue concentration, for example, bladder tissue concentration, at the C_{trough} ranges from about 100 to about 297 to about 500-fold (for example, about 100, 150, 200, 250, 300, 350, 400, or 500-fold) above the EC₅₀ of the antibody, or antigen-binding fragment thereof, wherein the EC₅₀ is about 0.021±0.020 μg/mL.

In some instances, the polyomavirus infection comprises a JC virus and the tissue concentration at the C_{trough} is at least about 29 to about 110 to about 158 to about 547-fold (for example, 20, 30, 40, 50, 60, 70, 80, 90, 100, 110, 120, 130, 140, 150, 200, 250, 300, 350, 400, 450, 500, 550, 600-fold) above the EC₅₀ of the antibody, or antigen-binding fragment thereof, wherein the EC₅₀ is about 0.215±0.130 μg/mL.

The EC₅₀ and C_{trough} levels of a polyomavirus neutralizing antibody against a BK virus genotype or JC virus can be determined according to routine techniques in the art. For example, C_{trough} levels can be measured by a validated sandwich ELISA-based assay using colorimetric detection, among other techniques, and the EC₅₀ can be measured in an ex vivo cell-based assay (see, for instance, Example 2).

For in vivo use, as noted above, for the treatment of human disease, the antibodies, antigen-binding fragments thereof, and other agents described herein are generally incorporated into one or more pharmaceutical or therapeutic compositions prior to administration.

Thus, certain embodiments relate to pharmaceutical or therapeutic compositions that comprise a therapeutically-effective amount or dose of a polyomavirus neutralizing antibody, or antigen-binding fragment thereof, as described herein. In some instances, a pharmaceutical or therapeutic composition described herein comprises polyomavirus neutralizing antibody, or antigen-binding fragment thereof, in combination with a pharmaceutically- or physiologically-acceptable carrier or excipient. In certain embodiments, a carrier comprises histidine and/or glycine, a saccharide such as sucrose, and/or a polyol such as a polysorbate. In specific embodiments, the carrier comprises histidine, sucrose, and a polysorbate.

In some embodiments, the pharmaceutical compositions described herein do not significantly form aggregates, have a desired solubility, and/or have an immunogenicity profile that is suitable for use in humans, as known in the art. Thus, in some embodiments, a composition comprising a polyomavirus neutralizing antibody, or antigen-binding fragment thereof, has about or less than about 50, 45, 40, 35, 30, 25, 20, 15, 14, 13, 12, 11, 10, 9, 8, 7, 6, 5, 4, 3, 2, or 1% aggregates, as measured, for example, by dynamic light scattering. Some compositions comprise a polyomavirus neutralizing antibody, or antigen-binding fragment thereof, that is at least about 50%, about 60%, about 70%, about 80%, about 90% or about 95% monodisperse with respect to the apparent molecular mass of non-aggregated antibody, or antigen-binding fragment thereof.

Administration of a composition may be achieved by a variety of different routes, including parenteral and enteral administration. Examples of parenteral administration include intravenous, subcutaneous, intrathecal, epidural, intracerebral, intracerebroventricular, intranasal, intramuscular, intra-arterial, and inhalational administration. Examples of enteral administration include oral or rectal administration. Particular embodiments include administration by IV infusion, for example, by intravenous bolus injection, by intravenous infusion (for example, over an approximately 10-90 minute period, or about 10, 20, 30, 40, 50, 60, 70, 80, or 90 minute period), or by escalating or continuous intravenous administration, for example, via an infusion pump or an ambulatory infusion device.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically-effective amount of an agent described herein, for treatment of a disease or condition of interest.

In certain embodiments, the dosage of a polyomavirus neutralizing antibody, or antigen-binding fragment thereof, for example, the dosage in step (a), is about 1 to about 100 mg/kg, or about 10-30 mg/kg, or about 1, 2, 3, 4, 5, 6, 7, 8, 9, 10, 15, 20, 25, 30, 35, 40, 45, 50, 60, 70, 80, 90, or 100 mg/kg. In some embodiments, the further dosage in step (c) is the same as or different than the dosage in (a), for example, at about 1 to about 100 mg/kg, or about 10-30 mg/kg, or about 1, 2, 3, 4, 5, 6, 7, 8, 9, 10, 15, 20, 25, 30, 35, 40, 45, 50, 60, 70, 80, 90, or 100 mg/kg. In some embodiments, the time between (a) and (c) is about, at least about, or no more than about 1, 2, 3, 4, 5, 6, 7, 8, 9, 10, 11, 12, 13, 14, 15, 15, 17, 18, 19, 20, 21, 22, 23, 24, 25, 26, 27, 28, 29, or 30 days, or 1, 2, 3, 4, 5, 6, 7, or 8 weeks, or 1, 2, or 3 months.

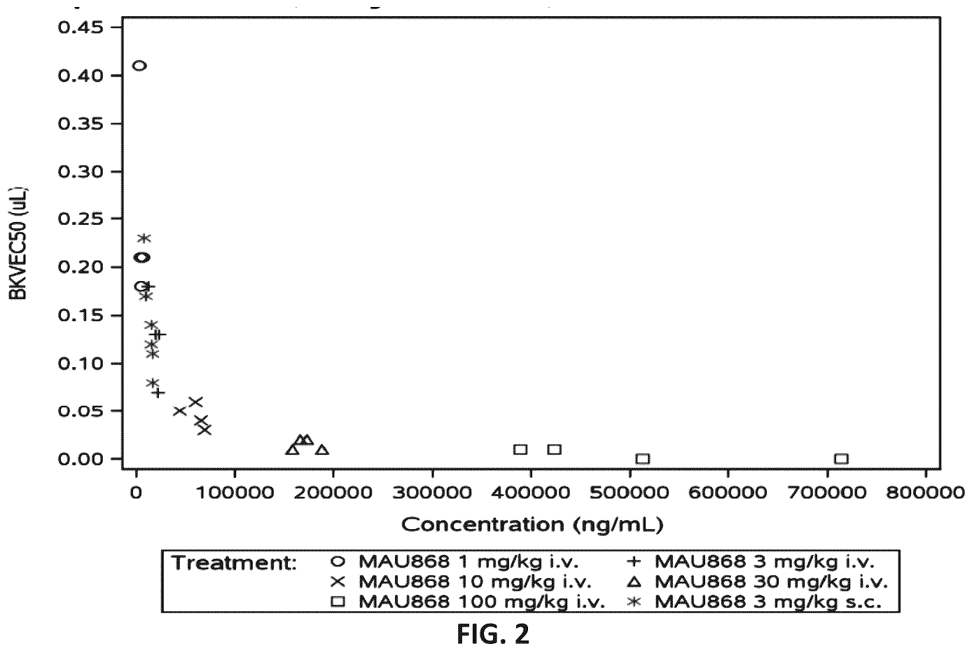

In some embodiments, the dosage of a polyomavirus neutralizing antibody, or antigen-binding fragment thereof, is about 10-30 mg/kg, or about 10-25, 10-20, 10-15, 15-30, 15-25, 15-20, 20-30, or 25-30 mg/kg, or about 10, 11, 12, 13, 14, 15, 16, 17, 18, 19, 20, 21, 22, 23, 24, 25, 26, 27, 28, 29, or 30 mg/kg. In some instances, the 10-30 mg/kg dosage provides optimal neutralizing activity (IC₅₀) by the subject's serum against the BK or JC polyomavirus, as measured in an in vitro or ex vivo viral assay (see, for example, FIG. 2, where neutralizing activity increased up to the dose of 30 mg/kg, evidencing that maximum neutralizing activity was achieved at doses >10 mg/kg to <30 mg/kg). Exemplary in vitro or ex vivo assays for measuring BK or JC viral replication include the use of primary renal proximal tubule epithelial (RPTE) cells grown and maintained in RPTE cell medium, as described, for example in Abend et al. (J Virol. 81 (1): 272-279, 2007) and Low et al. (Virology. 323:182-8, 2004). In specific embodiments, the dosage is administered intravenously or subcutaneously.

In certain embodiments, the dosing regimens and methods described herein are characterized by one or more pharmacokinetic profiles (see the Examples, including Table E2). For instance, following administration of a polyomavirus neutralizing antibody, or antigen-binding fragment thereof, at least 1, 2, 3, 4, 5, 6, 7, 8, 9, 10, 11, 12, 13, 14, 15, 16, 17, 18, 19, or 20% or more of the serum, circulating concentration of the antibody, or antigen-binding fragment thereof, penetrates the interstitial spaces of an infected organ, that is, an organ infected by a BK virus or JC virus. Examples of infected organs include the bladder, the kidneys, and the brain. In some embodiments, the mean clearance of the antibody, or antigen binding fragment thereof, is about 0.0760-0.0996 mL/day/kg. In some embodiments, the mean volume of distribution of the antibody, or antigen binding fragment thereof, is about 49.8-81.9 mL/kg.

Also included are combination therapies, for example, wherein a polyomavirus neutralizing antibody, or antigen-binding fragment thereof, is administered in combination with an additional agent, such as an immunosuppressive agent. Certain pharmaceutical or therapeutic compositions thus further comprise an additional agent, for example, an immunosuppressive agent. Examples of immunosuppressive agents include monophosphate dehydrogenase inhibitors, purine synthesis inhibitors, calcineurin inhibitors, mTOR inhibitors, mycophenolate mofetil (MMF), mycophenolate sodium, azathioprine, tacrolimus, sirolimus and cyclosporine.

The combination therapies described herein may include administration of a single pharmaceutical dosage formulation, which comprises a polyomavirus neutralizing antibody, or antigen-binding fragment thereof, and an additional agent, as well as administration of compositions comprising a polyomavirus neutralizing antibody, or antigen-binding fragment thereof, and an additional agent each in its own separate pharmaceutical dosage formulation. For example, a polyomavirus neutralizing antibody, or antigen-binding fragment thereof, and an additional agent can be administered to the subject together in a single dosage composition, or each agent administered in separate dosage formulations. For instance, a polyomavirus neutralizing antibody, or antigen-binding fragment thereof, and additional therapeutic agent can be administered to the subject together in a single parenteral dosage composition such as in a saline solution or other physiologically acceptable solution, or each agent administered in separate parenteral dosage formulations. Where separate dosage formulations are used, the compositions can be administered at essentially the same time (i.e., concurrently), or at separately staggered times (i.e., sequentially) and in any order. Combination therapy is understood to include all these regimens.

In certain embodiments, the dosage regimens or methods of treatment described herein reduce BK or JC viremia and/or viruria in the subject, for example, by about or at least about 10%, 20%, 30%, 40%, 50%, 60%, 70%, 80%, 90%, 100%, 200%, 300%, 400%, 500%, 1000%, 2000%, 3000%, 4000%, or 5000% or more, relative to a control (for example, relative to no composition or prior to treatment with a polyomavirus neutralizing antibody, or antigen-binding fragment thereof). In some embodiments, the dosage regimens or methods of treatment described herein reduce or improve one or more BK or JC virus-related symptoms in the subject, for example, symptoms such as blurred vision or other vision changes, brown or red urine, pain while urinating, reduced kidney function, difficulty urinating, cough, colds, trouble breathing, fever, muscle pain, muscle weakness, and/or seizures.

### EXAMPLES

### Example 1

### Pre-Clinical Characterization of MAU868, a Broadly Neutralizing Antibody Against BK Virus

Assays were performed to characterize the in vitro binding and virus neutralizing activities of MAU868. Binding affinity was determined using a solution equilibrium titration assay. Neutralization of BKV infection in primary renal proximal tubule epithelial cells (RPTE) was evaluated by quantitating TAg-expressing cells using an immunofluorescence-based high content imaging assay. The emergence of BKV resistance-associated variants (RAVs) with reduced susceptibility to MAU868 was investigated in two long-term selection studies with BKV genotypes I and IV in RPTE and HEK-293 cells. Crystallographic studies were conducted using the MAU868 single-chain variable fragment (scFv) bound to VP1 pentamers.

MAU868 had pM binding affinity and sub-nM neutralizing activity against the 4 major BKV genotypes, with EC₅₀ and EC₉₀ values ranging from 0.009 to 0.093 μg/ml (0.062 to 0.645 nM) and 0.102 to 4.160 g/ml (0.708 to 28.865 nM), respectively (see, for example, Table E1 below).

TABLE E1
In vitro binding affinity and neutralizing activity of
MAU868 across BKV genotypes

| Genotype | KD (pM) | EC₅₀ (μg/mL) |
|---|---|---|
| BKV genotype I* | 5.8 ± 1.8 | 0.009 ± 0.010 |
| BKV genotype II | 2.8 ± 0.6 | 0.040 ± 0.025 |
| BKV genotype III | 8.4 ± 3.7 | 0.093 ± 0.057 |
| BKV genotype IV* | 4.1 ± 1.3 | 0.021 ± 0.020 |

*BKV serotypes I and IV comprise about 95% of global seroprevalence.
Data presented as arithmetic mean ± standard deviation across replicates (n = 4 for KD; n = 3 for EC50).

No cytotoxicity was observed up to the highest concentration tested (500 μg/ml). MAU868 also potently neutralized BKV variants constructed to contain VP1 sequences from clinical isolates or highly prevalent VP1 polymorphisms, and JC virus, a related polyomavirus. No RAVs were identified following serial passage of BKV in the presence of MAU868 for up to 182 days. The crystal structure of MAU868 in complex with the VP1 pentamer at 2.66 Å resolution identified a conformational epitope including 3 contact residues in VP1 (Y169, R170, K172) that are strictly conserved across BKV isolates and account for the broad-spectrum activity of MAU868 and its high barrier-to-resistance. BKV variants with double or triple alanine substitutions at residues Y169, R170, or K172 were non-viable.

The potent, broad-spectrum antiviral activity combined with its high in vitro barrier-to-resistance demonstrate the potential for MAU868 as a first-in-class therapeutic agent for the treatment or prevention of BKV-associated diseases.

### Example 2

### In-Human Investigation of MAU868

A randomized, blinded, placebo-controlled, single ascending dose study was conducted at a single center in the United States. Study participants, investigators, and the sponsor were blinded. MAU868 was administered i.v. (1, 3, 10, 30, and 100 mg/kg) or s.c. (3 mg/kg) to healthy adults in a randomized, placebo-controlled, blinded, single ascending dose design. Each i.v. cohort was 5 subjects (4 MAU868:1 placebo); the s.c. cohort was 8 subjects (6 MAU868:2 placebo). Subjects were observed for 24 hours and followed for 106 days with routine safety monitoring and pharmacokinetic (PK) assessments.

MAU868 concentrations in serum were determined by a validated sandwich ELISA-based assay using colorimetric detection. MAU868 plasma concentration-time data were analyzed using standard non-compartmental methods using WinNonlin to generate the typical measures of exposure (AUC, Cmax, Tmax, elimination half-life). Dose proportionality was examined by regression of log-transformed Cmax and AUC versus log-transformed dose.

Ex vivo neutralizing activity of serum was measured before and 4 weeks after dose administration, using primary renal proximal tubule epithelial (RPTE) cells that were grown and maintained in RPTE cell medium.

Thirty-three (33) subjects completed the study. Adverse events were mild and infrequent; those occurring in more than one subject included nasal congestion (3, 9.1%), oropharyngeal pain (3, 9.1%), and injection site hemorrhage (ecchymosis after s.c. injection; 2, 6.1%). There were no infusion reactions. No subject discontinued the study due to an adverse event or developed anti-drug antibodies.

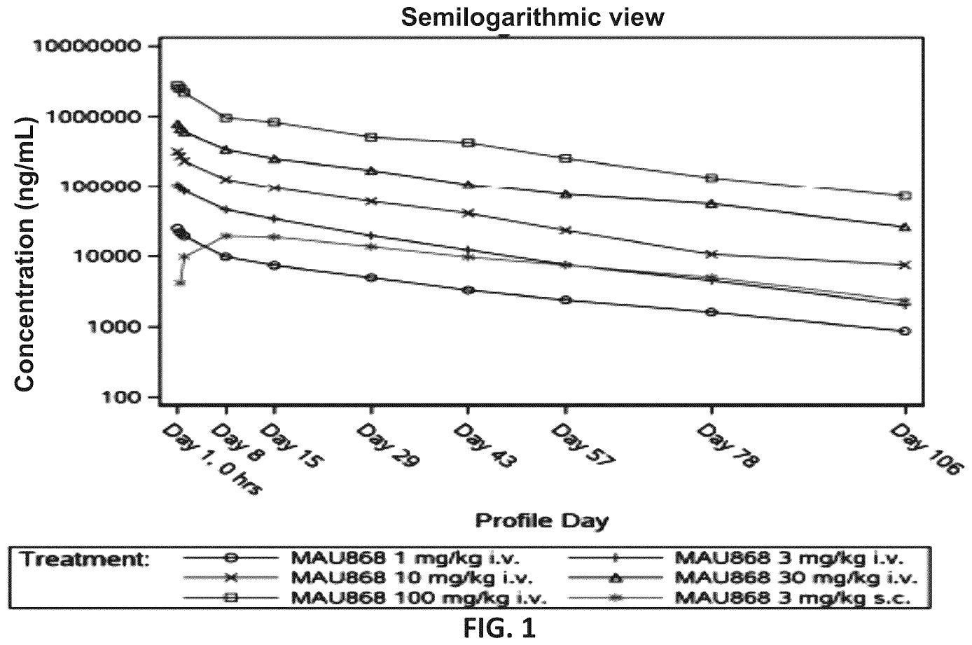

Exemplary PK characteristics are shown in FIG. 1 and Table E2 below.

TABLE E2
Summary of in vivo exposure to MAU868 by treatment group

| | 1 mg/kg | 3 mg/kg | 10 mg/kg | 30 mg/kg | 100 mg/kg | 3 mg/kg |
|---|---|---|---|---|---|---|
| Parameter | i.v. (n = 4) | i.v. (n = 4) | i.v. (n = 4) | i.v. (n = 4) | i.v. (n = 4) | s.c. (n = 6) |
| Cₘₐₓ | 24.7 ± 2.77 | 105 ± 7.92 | 316 ± 14.2 | 791 ± 271 | 2740 ± 353 | 20.1 ± 4.94^{§} |
| (μg/mL) | | | | | | |
| AUCₗₐₛₜ* | 9880 ± 2680 | 39500 ± 8310 | 115000 ± 13600 | 332000 ± 47400 | 1060000 ± 38400 | 22700 ± 6330 |
| (μg*hr/mL) | | | | | | |
| t_{1/2} (days) | 30.4 ± 11.4 | 24.8 ± 5.21 | 22.9 ± 1.80 | 26.1 ± 3.68 | 24.7 ± 8.54 | 30.0 ± 7.25 |
| C_{trough}\hat{ } | 4.94 ± 1.35 | 19.4 ± 5.02 | 59.9 ± 11.0 | 171 ± 127 | 510 ± 146 | 136 ± 367 |
| (μg/mL) | | | | | | |

Data presented as arithmetic mean ± standard deviation.
^{§}The Cₘₐₓ following subcutaneous administration occurred between 7 and 14 days after dose administration.
*AUCₗₐₛₜ = area under the serum concentration versus time curve until the last measurable concentration in each individual study participant. AUClast across treatment groups represented ≥90% of the calculated AUCinf, suggesting the study sampling duration was sufficient to characterize the duration of exposure to MAU868 following both routes of administration.
\hat{ }C_{trough} = serum concentrations measured on Day 29 (assuming a dosing regimen of every 4 weeks).

MAU868 PK included a half-life of 23 to 30 days. AUC and Cmax were dose-proportional, ranging from 9880 to 1060000 μg*hr/mL and 24.7 to 2740 μg/mL, and there was no evidence of FcRn saturation. Day 29 plasma MAU868 concentrations, adjusted for extravascular distribution to estimate parenchymal exposure, were approximately 7- to 751-fold higher than the highest in vitro EC₅₀ (0.093 μg/mL). Following i.v. administration, the mean clearance of MAU868 ranged between 0.0760 and 0.0996 mL/day/kg; the mean volume of distribution ranged between 49.8 and 81.9 mL/kg. For both routes of administration, the coefficients of variation for AUC and Cmax were ≤28% and ≤34%, respectively Bioavailability after s.c. injection was 57.6%. Maximum ex vivo neutralizing activity of serum was achieved for doses >10 mg/kg (see FIG. 2), and appeared optimal in the 10-30 mg/kg dosing range.

MAU868 was safe and well tolerated. The PK data evidence a dosing regimen for maintaining optimal serum/tissue concentrations of MAU898, for example, by monitoring and defining a minimum C_{trough}, and the ex vivo neutralizing activity suggests where the optimal therapeutic range may be for the treatment or prevention of BKV disease.

1-25. (canceled)

26. A method for treating a BK or JC polyomavirus infection in a human subject in need thereof, comprising intravenously administering to the subject a dosage of an antibody, or an antigen-binding fragment thereof, which specifically binds to a VP1 protein of the polyomavirus, wherein the dosage is between about 10 mg/kg and about 100 mg/kg,
wherein the antibody, or antigen-binding fragment thereof, comprises
a heavy chain variable (V_{H}) that comprises complementary determining region (CDR) V_{H}CDR1, V_{H}CDR2, and V_{H}CDR3 sequences of SEQ ID NOs: 6-8, respectively; and
a light chain variable (V_{L}) region that comprises V_{L}CDR1, V_{L}CDR2, and V_{L}CDR3 sequences of SEQ ID NOs: 9-11, respectively,
and variants thereof which specifically bind to the VP1 protein.

27. (canceled)

28. The method of claim 26, wherein
the V_{H} region comprises a sequence at least 80, 85, 90, 95, 97, 98, 99, or 100% identical to SEQ ID NO: 12, optionally wherein the V_{H} sequence has 1, 2, 3, 4, 5, or 6 alterations in the framework regions, optionally selected from one or more of V5Q, G9P, T10G, N30S, N30K, and N30Q; and
the V_{L} region comprises sequence at least 80, 85, 90, 95, 97, 98, 99, or 100% identical to SEQ ID NO: 13, optionally wherein the V_{L} sequence has 1, 2, 3, 4, 5, or 6 alterations in the framework regions.

29. The method of claim 26, wherein the V_{H} region comprises, consists, or consists essentially of SEQ ID NO: 12 and the V_{L} region comprises, consists, or consists essentially of SEQ ID NO: 13.

30. The method of claim 26, wherein the polyomavirus infection comprises a BK virus selected from one or more of genotype I, II, III, and IV.

31. The method of claim 26, wherein the polyomavirus infection comprises a JC virus.

32. The method of claim 26, wherein the subject is immuno-compromised.

33. The method of claim 26, wherein the subject is about to undergo, is undergoing, or has undergone a transplant procedure, optionally an organ transplant or cell-based transplant procedure.

34. The method of claim 33, wherein the transplant procedure is selected from a kidney transplant and a hematopoietic cell transplant (HCT).

35. The method claim 26, wherein the subject has or is at risk for having a condition selected from BK virus-associated nephropathy, BK virus-associated hemorrhagic cystitis, and JC virus-associated progressive multifocal leukoencephalopathy.

36. The method of claim 26, wherein at least 1, 2, 3, 4, 5, 6, 7, 8, 9, 10, 11, 12, 13, 14, 15, 16, 17, 18, 19, or 20% or more of the serum, circulating concentration of the antibody, or antigen-binding fragment thereof, penetrates an infected organ.

37. The method of claim 36, wherein the infected organ is a bladder or a kidney.

38-55. (canceled)

56. The method of claim 26, wherein the dosage is between about 10 mg/kg and about 30 mg/kg

57. The method of claim 26, wherein the dosage is between about 20 mg/kg.

57. The method of claim 26, wherein the dosage is about 100 mg/kg.

58. The method of claim 26, wherein the dosage of the antibody or antigen-binding fragment thereof is a first dosage of the antibody or antigen-binding fragment thereof, and the method further comprises administering a second dosage of the antibody or antigen-binding fragment thereof between about 28 days and about 30 days after the first dosage of the antibody or antigen-binding fragment thereof.
